(19) United States
(12) Patent Application Publication
(10) Pub. No.: US 2018/0228513 A1
(43) Pub. Date: Aug. 16, 2018

(54) METHOD FOR PRODUCING AN OCCLUSIVE BARRIER FOR BONE REGENERATION AND AN OCCLUSIVE BARRIER OBTAINED BY MEANS OF SAID METHOD

(71) Applicant: Mauricio Alberto LIZARAZO ROZO, Barakaldo (vizcaya) (ES)

(72) Inventor: Mauricio Alberto LIZARAZO ROZO, Barakaldo (vizcaya) (ES)

(21) Appl. No.: 15/511,218

(22) PCT Filed: Oct. 27, 2016
(86) PCT No.: PCT/ES2016/070289

§ 371 Date: Mar. 14, 2017

(30) Foreign Application Priority Data
Apr. 23, 2015 (ES) P201500309

(51) Int. Cl. A61B 17/58; A61C 8/02; G06F 17/50; G05B 19/4099

(52) U.S. Cl. CPC A61B 17/58; A61C 8/0006; A61F 2/2846; G05B 19/4099; G06F 17/50

(57) ABSTRACT

Disclosed are a method for producing an occlusive barrier (1) for bone regeneration, and the occlusive barrier (1) obtained by means of said method. The method comprises: obtaining a tomography image; processing and digitising the tomography image; reducing the noise of the tomography image and converting same into a three-dimensional CAD file; importing the three-dimensional CAD file into CAD modelling software; defining an area corresponding to the occlusive barrier (1); generating a surface corresponding to the occlusive barrier (1); converting the occlusive barrier (1) into layers by means of CAM software; printing the occlusive barrier (1) in titanium according to the layers; subjecting the occlusive barrier (1) to a thermal treatment and to a surface sandblasting treatment; and examining the orifices (4), thicknesses and dimensions of the occlusive barrier (1) in an optical gauge.

### SECTOR OF THE TECHNIQUE

This invention relates to the industry dedicated to bone regeneration.

### PRIOR ART

At present, and for several years now, the treatment of choice to regenerate alveolar bone necessary for the placement of dental implants is called Guided Bone Regeneration (GBR). Guided Bone Regeneration (GBR) can also be defined as a bone regeneration technique by inhibition of soft tissue proliferation, by closure with a defect membrane, prior to filling it with bone grafts or fillings in order to keep the space open.

Grafts and bone fillings are purported to have a mechanical and biological function. In the bone-host graft interface, there is a complex relationship where multiple factors can intervene for a correct or for non-incorporation of the graft, among them graft vascularization, conservation techniques, local factors, systemic factors and mechanical properties (depending on the type, size and shape of the graft used). Adequate bone volume for osseointegration is essential for implant treatments. One of the prominent components of the stomatognathic system is the alveolar bone, which is an odonto-dependent structure, since it forms along with the dental elements, holds them while fulfilling their function and disappears once the teeth are lost.

Among the materials used for bone regeneration are described those for filling or grafting (biological products that fill the bone defects); and among these materials autologous grafts, allogeneic materials, xenogeneic, bone substitutes, guided bone regeneration techniques and the use of bone morphogenetic proteins are included.

In this sense, the various materials used can work with at least one of three known mechanisms or processes:
Osteogenesis: Synthesis of new bone from cells derived from the graft or host. Requires cells capable of generating bone.
Osteoinduction: This is a process of cell differentiation and recruitment, modulated by growth factors derived from the graft matrix, whose osteogenic activity is stimulated by extracting bone mineral.
Osteoconduction: It is a process by which the graft material provides a suitable environment, structure or physical material suitable for the apposition of new bone by a predictable pattern, determined by the graft biology and the mechanical environment of the graft-host interface.

Ideal bone grafts and fillings should give rise to these three processes, in addition to being biocompatible and providing mechanical stability. Biocompatibility can be defined when a material is considered compatible and only causes desired or tolerable reactions in the living organism.

In order to achieve some of the processes named above, bone grafts have been studied for more than four decades. Among the different options are:
A. Autologous or autogenous grafts: Bone obtained from the patient and for this reason there is little antigenic capacity. It is obtained from intraoral sites (chin, maxillary tuberosity, ascending branch) that are used for small defects or extra oral (iliac crest, tibia or calotte) when more is required. The choice of each approach will depend on the type, size and shape of the bone cavity, clinical experience and professional preference.
B. Allogeneic grafts or allografts: These are from individuals of the same species, but genetically different. They may be classified according to their processing as:
   Frozen allografts.
   Lyophilized allograft (freeze-dried).
   Freeze-dried and demineralized allografts.
   Irradiated bone.
   The advantages of the allograft include its availability in significant amounts, in different shapes and sizes, no sacrificing of host structures and no donor site morbidity. Disadvantages are related to the quality of the regenerated bone tissue, which is not always predictable. A process to eliminate their antigenic capacity is needed.
C. Heterologous grafts or xenografts: These are of natural origin, from another species (animal) and contain the natural minerals of the bone. For example, bovine bone and coral derivatives (Ostrix, Osteogen, Bio-oss, Interpore).
D. Alloplastic or synthetic grafts: These are synthetically manufactured materials. They are found in various shapes, sizes and textures. Biological bone responses will depend on the manufacturing techniques, crystallinity, porosity and degree of resorption.
   They may be: Ceramic: These are the most commonly used, for example synthetic calcium phosphate (hydroxyapatite and tricalcium phosphate). Polymers: Such as Bioplan, HTR. Bioactive ceramic glass: composed of calcium and phosphate salts, and sodium and silicon salts (Biogass, pedioglass, Biogran).

All implantation material should trigger a reaction that is as physiologically possible with the surrounding tissues. It is essential to know the normal biological processes that are triggered in the regeneration and the physical, mechanical and biological characteristics of each material.

At present, to use autologous grafts, a surgical procedure is required at the donor site for its production, with the consequent risk of postoperative morbidity, infection, pain, hemorrhage, muscle weakness, neurological injury, graft necrosis, among others; in addition, the surgical time is considerably increased and in some cases the amount of graft removed may be insufficient.

In the current technique of bone regeneration, the professional must perform by hand the device that he will implant in the patient. When the devices or barriers are preformed, spaces may remain that allow soft tissues to be invaded as well as the entrance of bacteria, with the consequent risk of infection and therefore of treatment failure.

Currently, the professional attends to the patient without any planning, which considerably limits his understanding of the surgical field that he will encounter.

In the current technique of guided bone regeneration (GBR) for bone regeneration, neoformation is expected as reabsorption or degradation of the graft or the filling used occurs, since its function is to maintain the space.

At present, in guided bone regeneration (GBR), depending on the bone material used as grafting or filling, the time required for the reabsorption of the material determines the formation of the new tissue. For example, in the case of Cerasorb® tricalcium phosphate (synthetic ceramic graft) the average time for resorption is 24 to 36 months; and in the case of Bio-oss® (heterologous graft of bovine bone), because it is a ceramic material, it is not absorbed but over time it forms a mixture between the filling material and bone.

Likewise, guided bone regeneration (GBR) requires a minimum of 12 to 24 months of waiting for bone formation, in order to be able to place implants, and leave them 6 months longer for their osseointegration, and thus proceed with the patient's dental rehabilitation.

Likewise, guided bone regeneration (GBR) requires a minimum wait of 12 to 24 months for bone formation, in order to be able to place implants, and leave them for 6 months more for their osseointegration, and then to be able to proceed with the patient's dental rehabilitation.

Currently, guided bone regeneration (GBR) processes which use flexible membranes that collapse under their own weight is responsible for a decrease in the volume of bone required in the regeneration.

In 2001, Koustopoulos stated: “The mere fact of filling a space with a biomaterial adjacent to the bone does not necessarily increase its formation but on the contrary, may inhibit it”; this demonstrates the unreliability of this current technique in the regeneration of alveolar bone, which is necessary and indispensable for the placement of implants.

### OBJECT OF THE INVENTION

Within biotechnological advances, alternatives to fillers and bone grafts have arisen, such as a titanium occlusion barrier, which is ideal because of its great osteoconductive capacity.

Occlusive barriers belong to the sector that involves additive techniques such as laser sintering and subtractive ones such as computerized machining applied in medical sciences such as dentistry.

In this way, the occlusive barrier is a biomedical device custom made for the patient, designed by computer and manufactured by titanium laser sintering technology, which adapts to the measurements of the anatomical structure of the patient. The object of the occlusive barrier is to create a space between the bone tissue and the gingival tissue to promote bone growth from the layer of stem cells that is covering the outer surface of the bone (endosteum). Its function, therefore, is to maintain the space to support the clot, which ultimately achieves tissue regeneration. This structure or biomedical device makes it possible to maintain stability of the clot and isolated it from the external environment, also avoiding bacterial invasion that would impede the regeneration process.

Accordingly, the occlusive barrier is printed on one or more pieces. With the occlusive barrier being formed of more than one piece, the occlusive barrier comprises a concavity in at least two of the pieces, each of the recesses being configured to partially enclose a tooth and so form the concavities that together surround the tooth. In this way, said pieces are complementary to jointly define at least one through hole into which the tooth may be fitted.

Biocompatibility is defined as the ability of a material to act with a suitable response to the host, in a specific application. This type of material is known as a biomaterial used for the service of medicine, in this case dentistry, to interact with biological systems inducing a specific biological activity.

The reasons for considering titanium as the ideal biomaterial in the making of custom occlusive barriers are:
Titanium is inert, the oxide covering in contact with the tissues is insoluble, so that no ions that might react with organic molecules are released.
Titanium in living tissues acts as a surface upon which bone will grow and adhere to the metal.

### DESCRIPTION OF THE FIGURES

FIG. 1 shows an occlusive barrier that is object of this invention according to a preferred embodiment.

FIG. 2 shows an occlusive barrier that is object of this invention according to another preferred embodiment.

FIG. 3 shows another view of the occlusive barrier shown in FIG. 1.

FIG. 4 shows a sectional view of the occlusive barrier according to the preferred embodiment shown in FIGS. 1 and 3.

FIGS. 5 to 7 show the occlusive barrier that is the object of this invention according to yet another preferred embodiment.

### DETAILED DESCRIPTION OF THE INVENTION

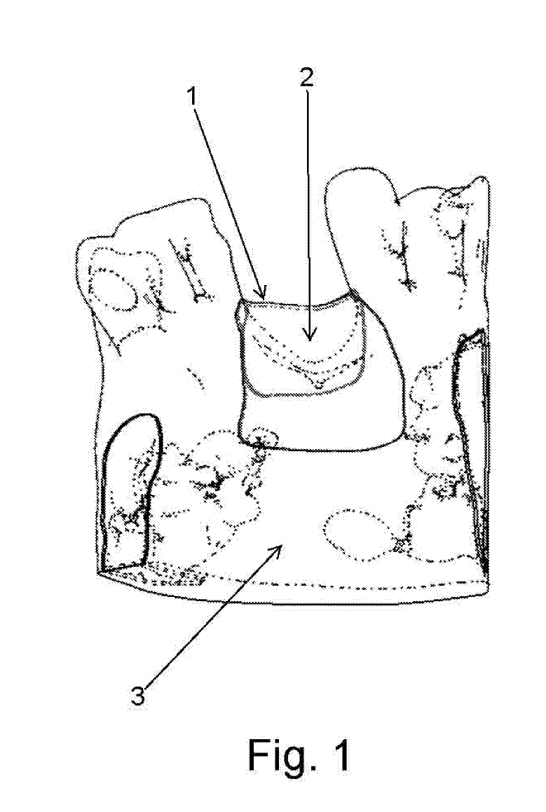

In FIGS. 1 to 3, an occlusive barrier (1), which is a biomedical device made of titanium, is individualized for each patient. The occlusive barrier (1) makes reconstruction of new bone tissue possible and/or is used to replace and regenerate destroyed or lost structures, and without the need to use filler materials or bone grafts.

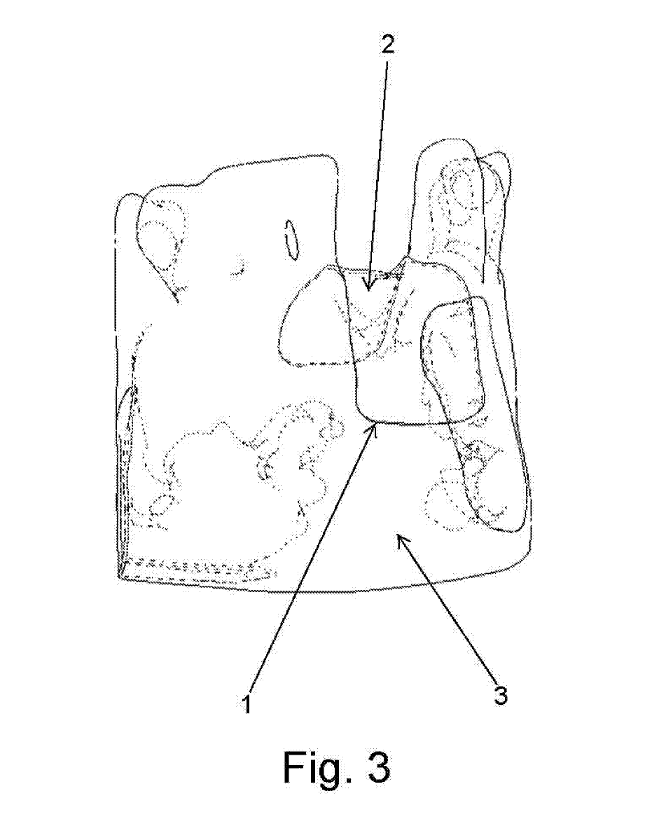

The customized occlusive barrier (1) as a biomedical device, induces new bone formation by acting as a biological barrier to prevent migration of epithelial cells from connective tissue and/or bacteria that would cause inhibition of bone growth. The occlusive barrier (1) is one hundred percent compatible, has an occlusive property because it is customized to the patient, since it is fully adapted to the surgical site, having the capacity to maintain a total regenerative space (2) and the possibility of vascularization. The occlusive barrier 1 is designed to ensure three-dimensional reconstruction of defects of the alveolar bone (3) and to facilitate restoration of alveolar bone (3) by appropriate fixation of the restitution material. The occlusive barrier (1) promotes adequate space (2) for the formation of a natural fibrin molding, a precursor of bone tissue, as may be seen in FIGS. 1 and 3.

The occlusive barrier (1) possesses the osteoconduction mechanism since it provides an environment, structure and physical material that triggers a three-dimensional growth of capillaries, perivascular tissue and most important, the recruitment of mesenchymal stem cells in the area, for its subsequent differentiation into osteoblasts modulated by growth factors. This scaffolding allows the formation of new bone through a predictable pattern, determined by the biology and dimensions of thickness and height previously given in the design and approved by the professional.

The design and production of the occlusive barrier (1) is described below. The process of developing customized barriers (1) involves a series of technological and technical processes ranging from the moment the diagnostic image is made in three dimensions, to the delivery of the biomedical device.

First, a tomography is obtained and it is then sent to the design laboratory. There, it is processed and digitized to determine the area to be treated, the noise is cleaned up and it is converted into a three-dimensional file that can be inserted into any CAD modeling software.

Once this CAD file is obtained, it is imported into the modeling software, where it is located and drawn upon, to define the area of the occlusive barrier (1) and to generate a surface that will become the occlusive barrier (1). Once approved and corrected by the doctors, the occlusive barrier (1) is exported for manufacturing.

For manufacturing, the exported design file is used and is made into a post-processed file in a CAM software, which converts it into layers for titanium printing. The occlusive barrier (1) is printed in layers of 30 or 60 microns thick and, once printed, is passed to a machining stage.

In this phase, the occlusive barrier (1) is initially subjected to a heat treatment to alleviate the molecular stress and to make the occlusive barrier (1) more ductile and strong, and then subjected to a surface sandblasting treatment which makes it possible for the occlusive barrier (1) to have the optimum characteristics for osseointegration. By sandblasting the surface a porosity is achieved to promote osteoconduction and the formation of blood vessels around the occlusive barrier (1). In this way, the occlusive barrier (1) with a mean arithmetic roughness (Ra) of 9-12 μm and a mean roughness range (Rz) of 40-80 μm is obtained.

Finally, the thicknesses, the orifices (4), and dimensions in the occlusive barrier (1) are checked in a highly accurate optical gauge, to ensure that the occlusive barrier (1) has the geometric characteristics initially defined. From there it goes to a sterilization treatment to be packed and sent to the customer.

The occlusive barrier (1) is subjected to an anodizing treatment. By means of this anodizing treatment, both organic and inorganic residues are cleaned from the surface, thus obtaining better resistance against corrosion, a decrease in the release of titanium ions to the physiological medium, greater surface hardness, improvement in the properties of osteoconduction and a coloration similar to that of the gums. Coloration is important in cases where the occlusive barrier (1) is exposed after placement in the patient to reduce the associated visual impact.

The thickness of the occlusive barrier (1) is between 0.3 and 0.6 millimeters. Lower values hinder the ability to maintain the space (2) and higher values make it difficult for the patient to accept the occlusive barrier (1) in the corresponding fixation area.

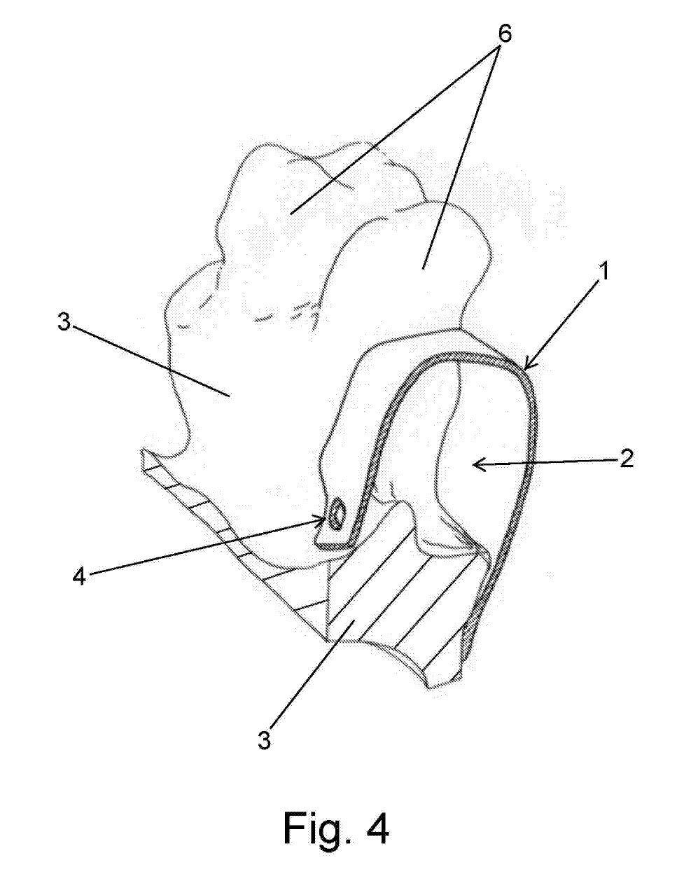

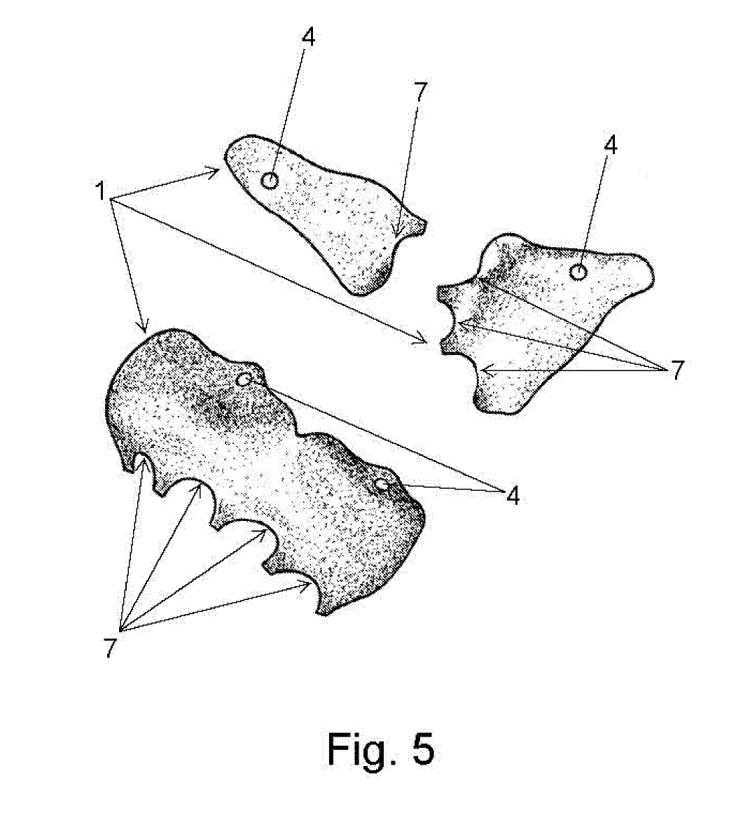

The orifices (4), which may be seen in FIGS. 4 and 5, are configured for the insertion of screws (5), preferably of titanium. The orifices (4) are for securing the occlusive barrier (1) to the patient's alveolar bone (3). For this reason, said orifices (4) are located in the occlusive barrier (1) depending on the patient, and more specifically on the available bone (3) of the patient.

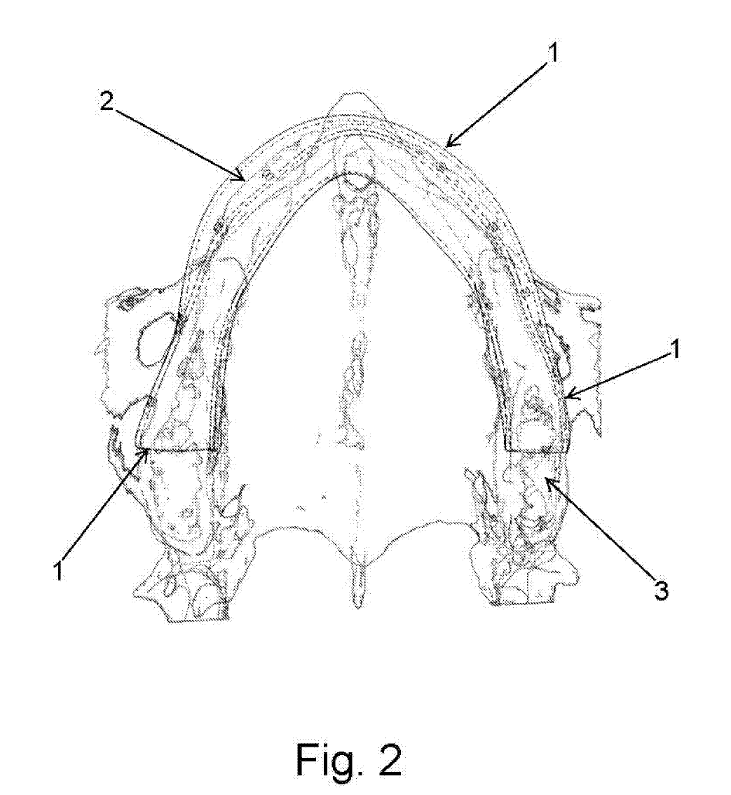

The occlusive barriers (1) may be partial or total. These (1) are defined as a function of their longitudinal extension upon the alveolar bone (3). Examples of the occluding barriers (1) may be seen in FIGS. 1 and 3 to 7 when they are partial and in FIG. 2 an example is shown of the occlusive barrier (1) when it is total. In this way, the partial occlusive barriers (1) may be limited by the teeth (6) at least at one of their longitudinal ends, while the total occlusive barriers (1) cover the entire longitudinal extension of the alveolar bone (3) in which the teeth (6) are located and upon which they are available. The longitudinal extent of the occlusive barriers (1) on the alveolar bone (3) may be greater than in the case of flexible membranes because these (1) do not collapse under their own weight as occurs with such membranes.

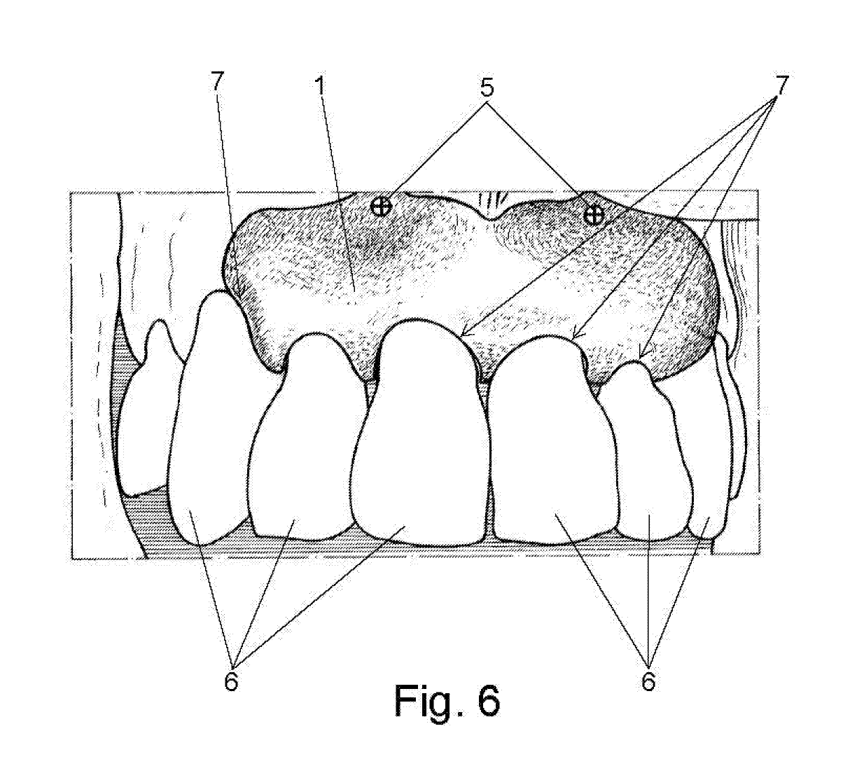

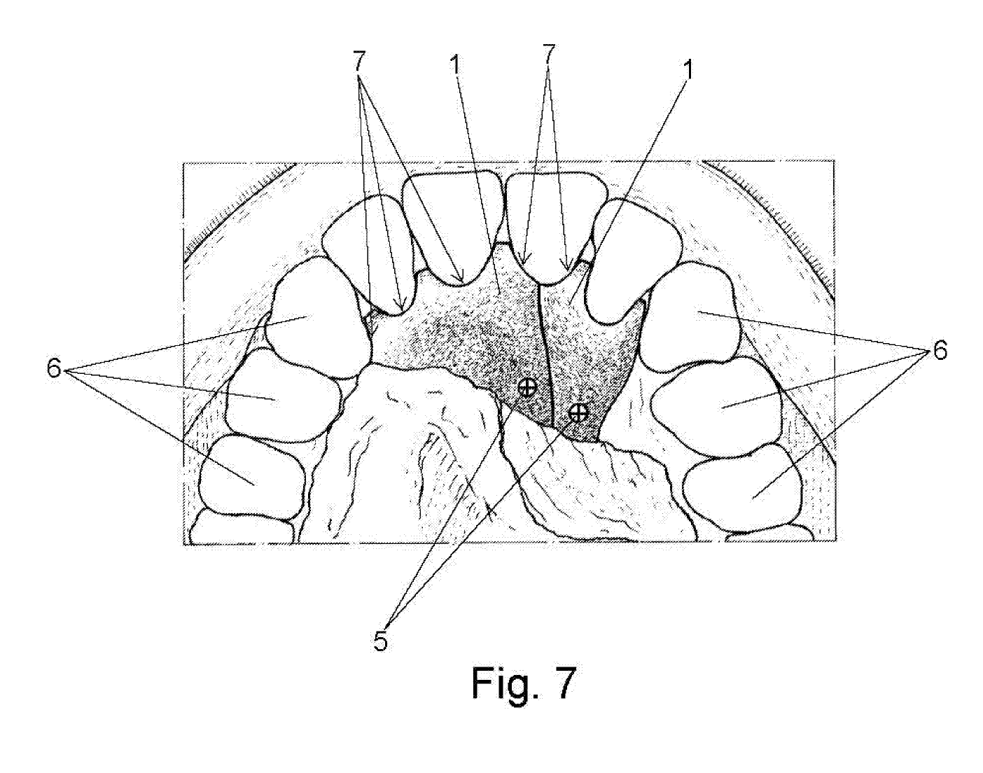

Also, in accordance with the design and the production described above, the occlusive barrier (1) can be printed in one piece, as is seen in FIGS. 1 to 3, or on several pieces, as is seen in FIGS. 5 to 7. The parts forming an example of the occlusive barrier (1), namely three, are shown in FIG. 5, while FIGS. 6 and 7 show the arrangement of said occlusive barrier (1) together.

Accordingly, the occluding barrier (1) is printed on one piece or on at least two pieces. With the occlusive barrier (1) formed by more than one piece, the occlusive barrier (1) comprises a concavity (7) in at least two of the pieces, each of these recesses (7) being configured to partially surround one of the teeth (6). In this case, the recesses (7) together define a through hole according to the outer contour of the tooth (6) to cover the bone (3) while taking the tooth (6) into account. The occlusive barrier (1) is printed on several pieces mainly because the patient, on occasion, despite suffering from the defect or bone wear, still retains the tooth (6) or the teeth (6) of the affected area. The present method of manufacturing makes it possible to obtain the pieces in the most optimal form, in accordance with each case.

The pieces forming the occlusive barrier (1) are obtainable in a complementary way to cover the all or part of the alveolar bone (3) leaving the space corresponding to the teeth (6) located in the area intended to house the occlusive barrier (1) free, further leaving the corresponding gap (2) free. In the exemplary embodiment shown in FIGS. 5 to 7, the occlusive barrier (1) is of three pieces. As can be seen in FIG. 7, the occlusive barrier (1) may also be formed of several pieces to better adapt to the shape of the bone (3) and facilitate their placement, that is to say it may be formed by the laterally complementary pieces to cover the affected area according to the longitudinal extension of the bone (3).

The titanium used in the creation of the occlusive barrier (1) is a material designed to interact safely and effectively with biological systems. Biomaterial-host interactions do not present any type of safety problem for the patient, i.e., it is one hundred percent compatible. The titanium used is preferably a titanium alloy called Ti64 or Ti6Al4V, having a density of 4.43 g/cm³.

### Properties of Medical Titanium:

Titanium is inert, the oxide covering in contact with the tissues is insoluble, so that no ions that might react with organic molecules are released.
Titanium in living tissue acts a surface upon which the bone grows and adheres to the metal, forming an ankylotic anchor, also called osseointegration. This reaction normally only occurs in materials called bioactive and is the best base for functional dental implants.
It has good mechanical properties; its tensile force is very similar to that of the stainless steel used in surgical prostheses that are load bearing. It is much stronger than dentine or any cortical bone, thus making it possible for the implants to withstand heavy loads.
This metal is soft and malleable which helps absorb shock loads.

Titanium is a biocompatible metal (biomaterial) because the body's tissues tolerate its presence without any allergic reactions from the immune system. This biocompatibility property of titanium coupled with its mechanical qualities of hardness, lightness and strength have made a large number of medical applications possible, not only dental implants, but also hip and knee prostheses, bone screws, anti-trauma plates, components for manufacturing heart valves and pacemakers, surgical instruments, etc.

Characteristics of the occlusive barrier (1) are therefore:
Cellular occlusion: The occlusive barrier (1) has the property of being isolated from the gingival tissue of the flap that opens during surgery, from the maturation of the fibrin clot in the wound space.
Space holding capacity: The occlusive barrier (1) has the ability to withstand its own collapse determined by its rigidity. That is, the occlusive barrier (1) possesses the physical property of being able to withstand its own collapse determined by its rigidity, guaranteeing the predetermined bone volume in the design of the biomedical device.
Tissue integration: The occlusive barrier (1) should become as integrated as possible to the tissue where it is placed.

This invention is characterized by requiring a single surgery at the receiver site of the occlusive barrier (1), with no need for bone filler or graft of any type. Because it does not require any fillings, the osteoconductive capacity of titanium allows the blood vessels to construct scaffolding for the osteogenic cells in the clot, giving the proper conditions for the growth of the new bone.

Likewise, in the absence of any filler, biological mechanisms do not require foreign body resorption. Therefore, the new bone formation is commenced once the occlusive barrier (1) is placed, i.e., the time needed for tissue regeneration is much shorter.

By the technology used in the design and manufacturing process of the biomedical device, it makes it possible to understand the anatomy of the surgical field in its three dimensions prior to the surgery, even making possible the operation in a virtual way.

As described, this invention provides the use of digitalized design and manufacturing processes (CAD-CAM), the software required for printing the occlusive barrier (1) customized for the patient. Since the occlusive barrier (1) is a custom-manufactured device, the adaptation and the peripheral seal will completely prevent the entry of soft tissue and bacteria, a situation that makes it possible to guarantee the success of the treatment.

This invention is characterized by the possibility of placing the implants at the same surgical moment as the occlusive barrier (1), so that new bone tissue is formed at the same time as the osseointegration thereof is carried out with the implants, leading to a very significant gain in time for the initiation of patient rehabilitation.

Due to the conditions for tissue treatment using the custom made occlusive barrier (1) for the patient manufactured in medical use titanium, we are able to guarantee the success of the treatment 100%, and it will also be in compliance with all conditions of technological and scientific capacity for its implementation, in addition to the necessary mandatory protocols to obtain the expected result.

1. Method for manufacturing an occlusive barrier (1) for bone regeneration, characterized in that it comprises the following steps:
obtaining a tomography;
processing and digitizing the tomography;
cleaning up the tomography noise;
converting the tomography into a three-dimensional CAD file;
importing the three-dimensional CAD file into a CAD modeling software;
defining an area corresponding to the occlusive barrier (1);
generating a surface corresponding to the occlusive barrier (1);
converting the occlusive barrier (1) into layers using CAM software;
printing the occlusive barrier (1) in titanium in the form of layers;
subjecting the occlusive barrier (1) to a heat treatment;
subjecting the occlusive barrier (1) to a surface sandblasting treatment; and
examining the orifices (4), thicknesses and dimensions of the occlusive barrier (1) with an optical meter.

2. Method for manufacturing according to claim 1, characterized in that the layers of the occlusive barrier (1) which are printed in titanium are 30 or 60 microns thick.

3. Method for manufacturing according to claim 1 or 2, characterized in that it further comprises subjecting the occlusive barrier (1) to a sterilization treatment.

4. Method for manufacturing according to any one of the preceding claims, characterized in that the occlusive barrier (1) is printed in one piece.

5. Method for manufacturing according to any of the claims 1 to 3, characterized in that the occlusive barrier (1) is printed in at least two pieces.

6. Method for manufacturing according to claim 5, characterized in that the parts are complementary to jointly define at least one through hole in which a tooth (6) may be fitted.

7. Occlusive barrier (1) for bone regeneration obtained by the method of manufacturing of any one of the preceding claims.
